(19) United States
(12) Patent Application Publication
(10) Pub. No.: US 2008/0118449 A1
(43) Pub. Date: May 22, 2008

(54) COSMETIC FORMULATIONS OF PICEATANNOL AND RETINOIDS AND METHODS OF USE THEREOF

(76) Inventor: Alvin Ronlan, Pompano Beach, FL (US)

Correspondence Address: Alvin Ronlan, 3414 Norfolk Street, Pompano Beach, FL, 33062, US

(21) Appl. No.: 11/562,250

(22) Filed: Nov. 21, 2006

(51) Int. Cl. A61Q 17/04; A61K 31/045

(52) U.S. Cl. 424/59; 514/739

(57) ABSTRACT

A cosmetic composition for skin care containing retinol and piceatannol is disclosed. In embodiments of the cosmetic composition piceatannol is preferably present in an amount of 0.001 to 10% by weight, and more preferably 0.1 to 1% by weight, based on the total weight of the composition. The piceatannol-containing cosmetic composition of the present invention alleviates adverse effects of retinol, such as skin irritation, and also enhances the skin care effect of retinol.

### FIELD OF THE INVENTION

The present invention concerns a cosmetic composition containing retinoids and piceatannol as well as derivatives thereof useful for skin care treatment for improving collagen synthesis without irritation.

### BACKGROUND OF THE INVENTION

Retinol (vitamin A) is an endogenous compound which occurs naturally in the human body and is essential for normal epithelial cell differentiation. Natural and synthetic vitamin A derivatives are well recognized as anti-wrinkle actives which help to reduce the subcutaneous effects of aging as wrinkling, leatheriness, looseness, roughness, dryness, and mottling (hyper pigmentation) (see, U.S. Pat. Nos. 4,603,146 and 4,877,805 to Kligman). It has been postulated that retinoids act by producing inflammation, which causes thickening of the epidermis (acanthosis), and local intercellular edema, leading to exfoliation.

In formulating products containing retinoids, much attention is directed toward providing compositions which deliver and retain optimal concentrations of the retinoid in the stratum corneum with minimum absorption into the systemic circulation. Furthermore, promoting user compliance with respect to chronic treatment regimens is also important. Current retinoid formulations, however, can be drying and irritating and result in excessive peeling. Such formulations may cause individuals to refrain from using retinoid products as frequently and copiously as is necessary for optimum benefit.

The present invention is based, in part, on the unexpected finding that compositions containing natural or synthetic retinoid compounds along with piceatannol, as an anti-inflammatory agent deliver the skin regulating benefits of retinoid compounds with much reduced dryness and/or irritation. These compositions have improved user acceptance and, thus, promote better user compliance with a concomitant overall improvement in skin regulating benefit.

Piceatannol (trans-3,4,3′,5′-tetrahydroxystilbene) is a polyphenol compound found in a variety of plants, often together with the structurally related compound resveratrol (trans-4,3′,5′-trihydroxystilbene). Both substances are synthesized in plants in response to fungal and other environmental stress, classifying them as phytoalexins. Piceatannol has been identified as the active ingredient of Melaleuca leucadendron (white tea tree)/Tsuruga, T., Chun, Y. T., Ebizuka, Y. & Sankawa, U. (1991) Biologically active constituents of Melaleuca leucadendron: inhibitors of induced histamine release from rat mast cells. Chem. Pharm. Bull. (Tokyo) 39: 3276-3278.), Cassia garretiana, (Asian legume) Inamori, Y., Kato, Y., Kubo, M., Yasuda, M., Baba, K. & Kozawa, M. (1984) Physiological activities of 3,3′,4,5′-tetrahydroxystilbene isolated from the heartwood of Cassia garrettiana CRAIB. Chem. Pharm. Bull. (Tokyo) 32: 213-218) and Rheum undulatum (Korean rhubarb) (Ko, S. K., Lee, S. M. & Whang, W. K. (1999) Anti-platelet aggregation activity of stilbene derivatives from Rheum undulatum. Arch. Pharm. Res. 22: 401-403 and Matsuda, H., Kageura, T, Morikawa, T, Toguchida, I., Harima, S. & Yoshikawa, M. (2000) Effects of stilbene constituents from rhubarb on nitric oxide production in lipopolysaccharide-activated macrophages. Bioorg. Med. Chem. Lett. 10: 323-327), which are used in traditional herbal medicine, and as the antileukemic compound in the seed of Euphorbia lagascae (Ferrigni, N. R., McLaughlin, J. L., Powell, R. G. & Smith, C. R., Jr. (1984) Use of potato disc and brine shrimp bioassays to detect activity and isolate piceatannol as the antileukemic principle from the seeds of Euphorbia lagascae. J. Nat. Prod. 47: 347-352.), which is used in folk medicine to treat cancer, tumors and warts. Teguo et al. (6) detected piceatannol in cell suspension cultures of Vitis vinifera (wine grapes) (Teguo, P. W., Decendit, S., Krisa, S., Deffieux, G., Vercauteren, J., and Mérillon, J. M. (2001) The accumulation of stilbene glycosides in Vitis vinifera cell suspension cultures. J. Nat. Prod. 59: 1189-1191).

Cosmetic compositions containing resveratrol have been described. See for example Pezzuto et al. (U.S. Pat. No. 6,414,037) disclose a method for preventing or treating a skin condition with a resveratrol containing composition, and Pillai et al. (U.S. Pat. No. 6,358,517) disclose a cosmetic skin care composition containing resveratrol in combination with selected retinoids. No cosmetic compositions employing piceatannol as the active agent have been described. The uniqueness of piceatannol and that the additional hydroxyl group in piceatannol is not a trivial extension of resveratrol is clearly demonstrated by scientific evidence, see for example, Ashikawa et al. in J. Immunol. Dec. 1, 2002;169(11):6490-7.

Since piceatannol and its derivatives are phenolic compounds, they act as potent antioxidants.

### SUMMARY OF THE INVENTION

The primary invention is the discovery of retinoid containing skin conditioning compositions having greatly improved skin compatibility containing:

(a) from about 0.001% to about 5% of a retinoid selected from the group consisting of retinol, retinyl esters, retinal, retinoic acid, a retinoic acid salt, a derivative or analog thereof, and combinations of any of the foregoing;

(b) from about 0.0001% to about 10% of piceatannol, dermatologically acceptable salts, esters, amides, prodrugs and analogs thereof, and combinations of any of the foregoing; and

(c) a cosmetically acceptable vehicle.

The term “conditioning” as used herein means prevention and treatment of dry skin, photodamaged skin, appearance of wrinkles, age spots, aged skin, acne, skin lightening, psoriasis, atopic dermatosis, increasing stratum corneum flexibility, controlling sebum excretion and generally increasing the quality of skin. The composition may be used to improve skin desquamation and cellular proliferation.

The presence of piceatannol in the inventive product substantially improves the performance of retinol or a retinyl ester, i.e., piceatannol substantially increases the ability of retinol or a retinyl ester to affect cellular proliferation. In mammalian systems, Piceatannol exerts antioxidative as well as anti-inflammatory effects that interfere with cytokine production and function, although the molecular targets remain elusive; however a substantial increase in skin benefit is realized when piceatannol is combined with retinol or a retinyl ester. In short, the present invention is based, at least in part, on the discovery of synergistic interaction between retinol or a retinyl ester and piceatannol.

According to the present invention, by virtue of including an effective amount of piceatannol into compositions containing retinol or a retinyl ester, the performance of the compositions is substantially improved.

The present invention also includes a method of improving or preventing the condition of dry skin, photodamaged skin, appearance of wrinkles, age spots, aged skin, acne, skin lightening, psoriasis, atopic dermatosis, increasing stratum corneum flexibility, controlling sebum excretion and generally increasing the quality of skin, which method includes applying to the skin the inventive composition. Compositions of the invention are intended for topical application to mammalian skin which is already dry, flaky, lined, wrinkled, aged, photodamaged, or the inventive compositions may be applied prophylactically to normal healthy skin to prevent or reduce the deteriorative changes.

The invention further includes a cosmetic method of controlling skin irritation, sting or inflammation which may be caused by retinoids. In this respect, the invention also includes cosmetic composition containing piceatannol in combination with retinoids.

### DETAILED DESCRIPTION OF THE INVENTION

### Overview and Definitions:

The term “piceatannol” is intended to mean either the cis-isomer of piceatannol, the trans-isomer of piceatannol, or a mixture of the two isomers. The term is also intended to include both the naturally occurring active agent and the compound as it may be chemically synthesized in the laboratory. Further, when the term “piceatannol” is used herein, it is intended to encompass dermatologically acceptable salts, esters, amides, prodrugs and analogs of piceatannol.

The term “treat” as in “to treat a skin condition” is intended to include (1) preventing the condition, i.e., avoiding any clinical symptoms of the condition, (2) inhibiting the condition, that is, arresting the development or progression of clinical symptoms, and/or (3) relieving the condition, i.e., causing regression of clinical symptoms.

By “dermatologically acceptable” is meant a material that is not biologically or otherwise undesirable, i.e., the material may be administered to an individual along with the selected active agent without causing any undesirable biological effects or interacting in a deleterious manner with any of the other components of the pharmaceutical composition in which it is contained. Similarly, a “dermatologically acceptable” salt or a “dermatologically acceptable” ester of an active agent as provided herein is a salt or ester which is not biologically or otherwise undesirable.

“Optional” or “optionally” means that the subsequently described circumstance may or may not occur, so that the description includes instances where the circumstance occurs and instances where it does not. For example, recitation of an additive as “optionally present” in a formulation herein encompasses both the formulation containing the additive and the formulation not containing the additive.

### Active Agents for Treatment:

The invention, as noted above, involves the use of piceatannol in order to prevent or treat skin conditions associated with the use of retinoids.

Piceatannol may be used in natural form, i.e., as isolated from grape skins, wine or other plant-derived compositions, or it may be used as chemically synthesized in the laboratory or as obtained commercially, e.g., from Biomol Research Laboratories, Inc. (Plymuth Meeting, Pa.). Preferred methods for obtaining piceatannol from a natural source is to extract the compound from Picea abies bark.

The active agent may be used in the form of a dermatologically acceptable salt, ester, amide, prodrug or analog or as a combination thereof. Salts, esters, amides, prodrugs and analogs of piceatannol may be prepared using standard procedures known to those skilled in the art of synthetic organic chemistry and pharmaceutical formulation. Preparation of esters involves functionalization of hydroxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature.

Preferred derivatives of cis- and trans-piceatannol are those in which one or more of the compounds' hydroxyl groups, typically the 3-hydroxyl group, is conjugated to a mono- or di-saccharide, generally the 1-position of a monosaccharide. Examples of saccharides which may be conjugated to the piceatannol molecule include, but are not limited to, glucose, galactose, maltose, lactose and sucrose. Cis-piceatannol glucoside and trans-piceatannol glucoside (astringin) are particularly preferred.

### Cosmetic Formulations:

In a preferred embodiment, the active agents are incorporated into a topical formulation containing a topical carrier that is generally suited to topical administration and comprising any such material known in the art. The topical carrier is selected so as to provide the composition in the desired form, e.g., as an ointment, lotion, cream, microemulsion, gel, oil, solution, or the like, and may be comprised of a material of either naturally occurring or synthetic origin. It is essential, clearly, that the selected carrier not adversely affect the active agent or other components of the topical formulation. Examples of suitable topical carriers for use herein include water, alcohols and glycols and other nontoxic organic solvents, glycerin, mineral oil, silicone, petroleum jelly, lanolin, fatty acids, vegetable oils, waxes, and the like.

Particularly preferred formulations herein are colorless, odorless solutions, lotions, creams, microemulsions and gels.

Various additives, known to those skilled in the art, may be included in the topical formulations of the invention. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives.

A skin permeation enhancer serves to facilitate passage of therapeutic levels of active agent to pass through a reasonably sized area of unbroken skin. Suitable enhancers are well known in the art and include, for example: 2-propanol and dimethyl isosorbide

Examples of solubilizers include, but are not limited to, the following: 1,3-butylene glycol, dipropylene glycol

Other active agents may also be included in the formulation, e.g., other anti-inflammatory agents, analgesics, antimicrobial agents, antifungal agents, antibiotics, vitamins, antioxidants, and sunblock agents commonly found in sunscreen formulations including, but not limited to, titanium dioxide, zink oxide, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In the preferred topical formulations of the invention, the active agent is present in an amount in the range of approximately 0.005 wt. % to 10 wt. % of the formulation, preferably in the range of approximately 0.01 wt. % to 5 wt. % of the formulation, more preferably in the range of approximately 0.1 wt. % to 5 wt. % of the formulation, and most preferably in the range of approximately 0.1 wt. % to 2 wt. % of the formulation.

### Utility:

The composition according to the invention is intended primarily as a product for topical cosmetic application to human skin, especially as an agent for conditioning, moisturizing and smoothening the skin, and preventing or reducing the appearance of lined, wrinkled or aged skin.

The formulations are administered topically, to the skin as an ointment, lotion, cream, microemulsion, gel, solution or the like, as described in the preceding section, within the context of a dosing regimen effective to bring about the desired result. The preferred dose of active agent is in the range of about 1 to 100 ml. Generally, a dosing regimen herein involves administration of the topical formulation at least once daily.

It is to be understood that while the invention has been described in conjunction with the preferred specific embodiments thereof, the foregoing description, as well as the examples which follow, are intended to illustrate and not limit the scope of the invention. Other aspects, advantages and modifications will be apparent to those skilled in the art to which the invention pertains.

All patents, patent documents, and publications cited herein are hereby incorporated by reference in their entireties.

### Experimental

The following examples are put forth so as to provide those of ordinary skill in the art with a complete disclosure and description of how to make and use the formulations of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in degree C and pressure is at or near atmospheric. All solvents, reagents and formulation components were of Pharmaceutical Grade.

### EXAMPLE 1

This example demonstrates that piceatannol reduces retinoid induced irritation and improves skin appearance in solution type formulations.

The cosmetic composition of Example 1 and comparative examples 1 and 2 wee prepared using the components listed in Table 1.

TABLE 1

| | | Comparative | Comparative |
|---|---|---|---|
| | Example 1 | Example 1 | Example 2 |
| Component | Wt. % | Wt. % | Wt. % |
| Dimethyl isosorbide | 25.0 | 25.0 | 25.0 |
| Dipropylene glycol | 74.4 | 74.6 | 74.8 |
| d-alpha-Tocopherol | 0.2 | 0.2 | 0.2 |
| Retinyl palmitate (1.7 m · IU/g) | 0.2 | 0.2 | 0 |
| trans-Piceatannol | 0.2 | 0 | 0 |

The composition was prepared as follows: d-alpha-tocopherol, retinyl palmitate (1.7 m. IU/g) and trans-piceatannol were dissolved in dimethyl isosorbide at 30° C., and when a clear solution had formed dipropylene glycol was added also at 30° C. The operation was carried out under a nitrogen blanket and all solutions were stored under nitrogen.

The skin irritant potential of the cosmetic compositions of Example 1 and Comparative Example 1 and 2 were determined using a modification of the human 4-h patch test (Basketter D A, et al., Food Chem Toxicol 1997,:35: 845-852), involving application of 0.2 ml of the composition to be tested on a 25 mm plain Hill Top Chamber containing a Webril pad (Hill Top Companies, Cincinatti, Ohio, USA) to the skin of the upper outer arm of 16 human volunteers. Both upper arms of the panelist were used. Patches were applied in a balanced random order.

After 24 hours, the patches were removed and then the test areas were marked with a marking pen. After each lapse of 24, 48 and 72 hours, the test areas were observed and treatment sites were assessed for the presence of irritation using a 4 point scale (Table 2) at 24, 48 and 72 h after patch removal

TABLE 2
Assessment of reactions

| Grading | Description of response |
|---|---|
| 0 | No reaction |
| + | Weakly positive reaction |
| | (usually characterized by mild |
| | erythema or dryness across |
| | most of the treatment site) |
| ++ | Moderately positive reaction |
| | (usually distinct erythema possibly |
| | spreading beyond the treatment site) |
| +++ | Strongly positive reaction (strong, |
| | often spreading erythema with oedema) |

The results are shown in Table 3.

TABLE 3
The skin irritant potential of the cosmetic compositions of Example
1 and Comparative Example 1 and 2

| | | Comparative | Comparative |
|---|---|---|---|
| Composition | Example 1 | Example 1 | Example 2 |
| Grading | 0 | + | 0 |
| (average of 16) | | | |

The addition of piceatannol clearly reduced the irritancy of retinyl palmitate.

The effects of the cosmetic compositions of Example 1 and Comparative Example 1 and 2 on skin wrinkle improvement were determined by computer aided laser profilometry.

It involved sixteen female subjects of 30 years and over. The cosmetic compositions of Example 1 and Comparative Examples 1 and 2 were applied on the face (area of 2×2 cm2) of the test subjects, once a day with dose of 0.05 g for 9 weeks. Then, replicas of their skin wrinkles were prepared using white plastic silicon precision moulding compound. The changes in the skin wrinkles of the replicas were detected with Skin Visiometer (SV600 manufactured by Courage+Khazaka electronic GmbH, Germany). Three-dimensional images of the replicas were analyzed with a CCD camera. The skin wrinkle improvement effect was determined as average roughness of the wrinkles (Rz) according to the following numerical formula I:

Rz=(R1+R2+ . . . +Rn−1+Rn)/Number of wrinkles(n) (I)

Wherein, Rn is the roughness of each wrinkle and n is the number of the wrinkles. The results are shown in Table 4 below.

TABLE 4
Skin wrinkle improvement effect (9 weeks)
average roughness of the panel of the wrinkles (Rz, micron)

| | | | Comparative | | | Comparative | | |
|---|---|---|---|---|---|---|---|---|
| Example 1 | | | Example 1 | | | Example 2 | | |
| T0 | T9 | Δ % | T0 | T9 | Δ % | T0 | T9 | Δ % |
| 125 | 37 | −70 | 133 | 59 | −56 | 129 | 124 | −4 |

As can be seen from the above, the height (roughness) of the wrinkles was decreased by 70% (p<0.01) after 9 weeks use of the cosmetic composition in Example 1 and by 56% (p<0.01) after 9 weeks use of the cosmetic composition in Comparative Example 1, which means that both Example 1 and Comparative Example 1 significantly improve skin roughness compared to the placebo (Comparative Example 2). The result also shows that piceatannol actually enhances the effect of retinol on wrinkle improvement. It also shows that the cosmetic composition of the present invention exhibits a visible effect on wrinkle improvement in a very short time.

### EXAMPLE 2

This example demonstrates that trans-(3,5,3′,4′-tetrahydroxystilbene)-3-O-beta-d-glucopyranoside (trans-astringin) reduces retinoid induced irritation and improves skin appearance in solution type formulations.

The cosmetic composition of Example 2 was prepared using the components listed in Table 5.

TABLE 5

| | Example 2 |
|---|---|
| Component | Wt. % |
| Dimethyl isosorbide | 25.0 |
| Dipropylene glycol | 74.4 |
| d-alpha-Tocopherol | 0.2 |
| Retinyl palmitate (1.7 m · IU/g) | 0.2 |
| trans-Astringin | 0.2 |

The composition was prepared following the procedure under Example 1.

The skin irritant potential of the cosmetic compositions of Example 2 compared to Comparative Example 1 and 2 were determined using the same technique described in Example 1 The results are shown in Table 6.

TABLE 6
The skin irritant potential of the cosmetic compositions of Example 2
and Comparative Example 1 and 2

| | | Comparative | Comparative |
|---|---|---|---|
| Composition | Example 2 | Example 1 | Example 2 |
| Grading | 0 | + | 0 |
| (average of 16) | | | |

The addition of piceatannol-glucoside (astringin) clearly reduced the irritancy of retinyl palmitate.

The effects of the cosmetic compositions of Example 2 on skin wrinkle improvement were determined in the same manner as described under Example 1. The results are shown in Table 7

TABLE 7
Skin wrinkle improvement effect (9 weeks)
average roughness of the panel of the wrinkles (Rz, micron)

| | | | Comparative | | | Comparative | | |
|---|---|---|---|---|---|---|---|---|
| Example 2 | | | Example 1 | | | Example 2 | | |
| T0 | T9 | Δ % | T0 | T9 | Δ % | T0 | T9 | Δ % |
| 129 | 43 | −67 | 133 | 59 | −56 | 129 | 124 | −4 |

As can be seen from the above, the height (roughness) of the wrinkles was decreased by 67% (p<0.01) after 9 weeks use of the cosmetic composition in Example 2, which means that it significantly improve skin roughness compared to the placebo (Comparative Example 2). The result also shows that astringin actually enhances the effect of retinol on wrinkle improvement. It also shows that the cosmetic composition of the present invention exhibits a visible effect on wrinkle improvement in a very short time.

### EXAMPLE 3

This example demonstrates that piceatannol reduces retinoid induced irritation and improves skin appearance in emulsion (lotion) type formulations.

The cosmetic composition of Example 3 and comparative examples 5 and 6 were prepared using the components listed in Table 8.

TABLE 8

| | | Comparative | Comparative |
|---|---|---|---|
| | Example 3 | Example 3 | Example 4 |
| COMPONENT | % WEIGHT | % WEIGHT | % WEIGHT |
| Petrolatum | 3.20 | 3.20 | 3.20 |
| 3-Methyl Glucose | 1.20 | 1.20 | 1.20 |
| Sesquistearate | | | |
| PEG-20 Methylglucose | 1.80 | 1.80 | 1.80 |
| sesquistearate | | | |
| Glyceryl Stearate (and) | 1.80 | 1.80 | 1.80 |
| PEG-100 Stearate | | | |
| Stearic acid | 1.60 | 1.60 | 1.60 |
| Cetyl alcohol | 1.20 | 1.20 | 1.20 |
| Cyclomethicone | 2.40 | 2.40 | 2.40 |
| Water (purified) | 76.53 | 76.73 | 76.93 |
| Carboxyvinyl polymer | 0.30 | 0.30 | 0.30 |
| Glycerin | 2.40 | 2.40 | 2.40 |
| Propylene glycol | 3.00 | 3.00 | 3.00 |
| Dimethicone/Dimethiconol | 1.60 | 1.60 | 1.60 |
| Sodium hydroxide (18%) | 1.97 | 1.97 | 1.97 |
| Preservative | 0.40 | 0.40 | 0.40 |
| d-alpha-Tocopherol | 0.20 | 0.20 | 0.20 |
| Retinyl palmitate | 0.20 | 0.20 | 0.00 |
| (1.7 m · IU/g) | | | |
| Piceatannol | 0.20 | 0.00 | 0.00 |

The lotion were prepared using standard procedures for cosmetic emulsions

The skin irritant potential of the cosmetic compositions of Example 3 and Comparative Example 3 and 4 were determined using the same technique described in Example 1

The results are shown in Table 9.

TABLE 9
The skin irritant potential of the cosmetic compositions of Example 3
and Comparative Examples 3 and 4

| | | Comparative | Comparative |
|---|---|---|---|
| Composition | Example 3 | Example 3 | Example 4 |
| Grading | 0 | + | 0 |
| (average of 16) | | | |

The addition of piceatannol clearly reduced the irritancy of retinyl palmitate in lotion type cosmetic preparations.

The effects of the cosmetic compositions of Example 3 on skin wrinkle improvement were determined in the same manner as described under Example 1. The results are shown in Table 10

TABLE 10
Skin wrinkle improvement effect (9 weeks)
average roughness of the panel of the wrinkles (Rz, micron)

| | | | Comparative | | | Comparative | | |
|---|---|---|---|---|---|---|---|---|
| Example 3 | | | Example 3 | | | Example 4 | | |
| T0 | T9 | Δ % | T0 | T9 | Δ % | T0 | T9 | Δ % |
| 131 | 59 | −55 | 128 | 88 | −31 | 127 | 112 | −12 |

As can be seen from the above, the height (roughness) of the wrinkles was decreased by 55% (p<0.01) after 9 weeks use of the cosmetic composition in Example 3, and by 31% (p<0.01) after 9 weeks use of the cosmetic composition in Comparative Example 3, which means that both Example 3 and Comparative Example 3 improve skin roughness compared to the placebo (Comparative Example 4). The result also shows that piceatannol actually enhances the effect of retinol on wrinkle improvement. It also shows that the cosmetic composition of the present invention exhibits a visible effect on wrinkle improvement in a very short time.

### EXAMPLE 4

This example demonstrates that piceatannol glucoside (astringin) reduces retinoid induced irritation and improves skin appearance in emulsion (lotion) type formulations.

The cosmetic composition of Example 4 was prepared using the components listed in Table 11.

TABLE 11

| | Example 4 |
|---|---|
| COMPONENT | % WEIGHT |
| Petrolatum | 3.20 |
| 3-Methyl Glucose Sesquistearate | 1.20 |
| PEG-20 Methylglucose sesquistearate | 1.80 |
| Glyceryl Stearate (and) PEG-100 Stearate | 1.80 |
| Stearic acid | 1.60 |
| Cetyl alcohol | 1.20 |
| Cyclomethicone | 2.40 |
| Water (purified) | 76.53 |
| Carboxyvinyl polymer | 0.30 |
| Glycerin | 2.40 |
| Propylene glycol | 3.00 |
| Dimethicone/Dimethiconol | 1.60 |
| Sodium hydroxide (18%) | 1.97 |
| Preservative | 0.40 |
| d-alpha-Tocopherol | 0.20 |
| Retinyl palmitate (1.7 m · IU/g) | 0.20 |
| Astringin | 0.20 |

The lotion were prepared using standard procedures for cosmetic emulsions

The skin irritant potential of the cosmetic compositions of Example 4 was determined using the same technique described in Example 1

The results are shown in Table 12.

TABLE 12
The skin irritant potential of the cosmetic compositions of Example 4
and Comparative Examples 3 and 4

| | | Comparative | Comparative |
|---|---|---|---|
| Composition | Example 3 | Example 3 | Example 4 |
| Grading | 0 | + | 0 |
| (average of 16) | | | |

The addition of astringin clearly reduced the irritancy of retinyl palmitate in lotion type cosmetic preparations.

The effects of the cosmetic compositions of Example 4 on skin wrinkle improvement were determined in the same manner as described under Example 1. The results are shown in Table 13

TABLE 13
Skin wrinkle improvement effect (9 weeks)
average roughness of the panel of the wrinkles (Rz, micron)

| | | | Comparative | | | Comparative | | |
|---|---|---|---|---|---|---|---|---|
| Example 4 | | | Example 3 | | | Example 4 | | |
| T0 | T9 | Δ % | T0 | T9 | Δ % | T0 | T9 | Δ % |
| 135 | 47 | −65 | 128 | 88 | −31 | 127 | 112 | −12 |

As can be seen from the above, the height (roughness) of the wrinkles was decreased by 65% (p<0.01) after 9 weeks use of the cosmetic composition in Example 3, which means that Example 4 improve skin roughness compared to the placebo (Comparative Example 4). The result also shows that astringin actually enhances the effect of retinol on wrinkle improvement and that astringin seems to be m ore efficient than piceatannol in emulsion type cosmetic preparations. It also shows that the cosmetic composition of the present invention exhibits a visible effect on wrinkle improvement in a very short time.

What is claimed:

1. A skin care composition comprising:
i) a retinoid in an amount from about 0.005 to about 5 wt %;
ii) piceatannol, dermatologically acceptable salts, esters, amides, prodrugs and analogues thereof, and combinations of any of the foregoing in an amount from about 0.001 to about 10 wt %
iii) a dermatologically acceptable vehicle; wherein said retinoid is present in an amount of about 0.005 to about 5 wt %

2. The method of claim 1, wherein the active agent is piceatannol.

3. The method of claim 2, wherein the active agent is a conjugate of piceatannol and a mono- or di-saccharide.

4. The method of claim 3, wherein the active agent is piceatannol-3-O-beta-d-glucopyranoside (astringin).

5. A composition according to claim 1, wherein the retinoid is selected from the group consisting of retinol, retinyl esters, retinal, retinoic acid, a retinoic acid salt, a derivative or analogue thereof, and combinations of any of the foregoing.

6. A composition according to claim 1, wherein said composition further comprises a compound selected from the group consisting of sunscreens and tocopherol anti-oxidants, and combinations of any of the foregoing.

7. A composition according to claim 1 which further comprises from about 1% to about 99.5% of a dermatological carrier.

8. A composition according to claim 7 in the form of an ointment, lotion, cream, emulsion, microemulsion, gel or solution.

9. A method for conditioning, moisturizing and smoothening the skin, and preventing or reducing the appearance of lined, wrinkled or aged skin comprising applying to the skin a safe and effective amount of the composition according to claim 1.
